(19) United States
(12) Patent Application Publication
(10) Pub. No.: US 2025/0275871 A1
(43) Pub. Date: Sep. 4, 2025

(54) DEVICES AND METHODS FOR IMPROVED REMOVAL OF CORTICAL MATERIAL

(71) Applicant: Alcon Inc., Fribourg (CH)

(72) Inventors: Francisco Javier Ochoa, La Mirada, CA (US); John Morgan Bourne, Irvine, CA (US); Satish Yalamanchili, Irvine, CA (US); Kevin Canzone, Corto de Caza, CA (US)

(21) Appl. No.: 19/010,443

(22) Filed: Jan. 6, 2025

Related U.S. Application Data
(60) Provisional application No. 63/559,264, filed on Feb. 29, 2024.

(51) Int. Cl. A61F 9/008; A61M 1/00

(52) U.S. Cl. CPC A61F 9/008; A61M 1/77; A61F 2009/00887

(57) ABSTRACT

Devices and methods are disclosed for improved removal of cortical material. In certain embodiments, an ophthalmic surgical instrument includes a shaft having a distal end configured to be inserted into an eye of a patient toward a treatment area, and a projection located at the distal end of the shaft, the projection having a distal surface that extends at an angle to a longitudinal axis of the shaft, the distal surface configured to be positioned facing the treatment area during use of the ophthalmic surgical instrument. The projection is located on a tip or a sleeve of the ophthalmic surgical instrument.

### INTRODUCTION

Cataract surgery involves removing a cataractous lens and replacing the lens with an artificial intraocular lens (IOL). The cataractous lens is typically removed by fragmenting the lens and aspirating the lens fragments out of the eye. The lens may be fragmented using, e.g., a phacoemulsification probe, a laser probe, or another suitable instrument. After the lens has been fragmented, a secondary irrigation/aspiration (I/A) handpiece and or a tip may be used to aspirate the fragments out of the eye, and to polish the lens capsule. The secondary I/A handpiece may also be used to irrigate fluid into the eye to maintain an intraocular pressure (IOP) and prevent collapse of the eye. A surgeon may therefore need to remove the phacoemulsification probe from the eye to switch fluidic lines between the phacoemulsification probe and the secondary I/A handpiece during the procedure to remove the fragmented lens and maintain stability of the eye's intraocular pressure (IOP) and anterior chamber (AC).

However, inserting and removing different ophthalmic surgical instruments throughout the course of a procedure can result in unwanted and unintentional trauma to ocular tissues, which can potentially lead to other ophthalmic complications. Further, having to switch between different ophthalmic surgical instruments throughout the procedure may increase the amount of time needed to complete the procedure, which further increases patient risk.

### BRIEF SUMMARY

The present disclosure relates generally to ophthalmic surgical instruments for removal of cortical material.

In certain embodiments, an ophthalmic surgical instrument is provided. The ophthalmic surgical instrument includes a shaft having a distal end configured to be inserted into an eye of a patient toward a treatment area, and a projection located at the distal end of the shaft, the projection having a distal surface that extends at an angle to a longitudinal axis of the shaft, the distal surface configured to be positioned facing the treatment area during use of the ophthalmic surgical instrument.

In certain embodiments, a method of performing an ophthalmic procedure is provided. The method includes inserting a distal end of an ophthalmic surgical instrument into an eye of a patient toward a treatment area, the ophthalmic surgical instrument comprising a shaft and a projection, performing a phacoemulsification procedure with the ophthalmic surgical instrument in the eye, and without withdrawing the distal end of the ophthalmic surgical instrument from the eye, performing capsule polishing with the projection.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A shows an example ophthalmic surgical system that may be used to perform ophthalmic procedures on an eye, according to certain embodiments.

FIG. 1B shows example components of a surgical console of the ophthalmic surgical system shown in FIG. 1A, according to certain embodiments.

FIG. 2 shows an example of an ophthalmic surgical instrument with a laser optical fiber, according to certain embodiments.

FIG. 3 shows an enlarged view of a distal end of the ophthalmic surgical instrument of FIG. 2, according to certain embodiments.

FIG. 4 shows a tip that can be used in conjunction with the ophthalmic surgical instrument of FIG. 2, according to certain embodiments.

FIG. 5 shows the distal end of an ophthalmic surgical instrument with the tip of FIG. 4, according to certain embodiments.

FIG. 6 shows another view of the distal end of the ophthalmic surgical instrument of FIG. 5, according to certain embodiments.

FIG. 7A shows an irrigation sleeve for the ophthalmic surgical instrument shown in FIG. 2, according to certain embodiments.

FIG. 7B shows the ophthalmic surgical instrument of FIG. 2 with the sleeve shown in FIG. 7A, according to certain embodiments.

FIG. 8A shows a distal end of an ophthalmic surgical instrument with an optical fiber disposed outside the shaft, according to certain embodiments.

FIG. 8B is a cross-sectional side view of the ophthalmic surgical instrument shown in FIG. 8A, according to certain embodiments.

FIGS. 9A-9B show the ophthalmic surgical instrument of FIGS. 8A-8B being inserted into an eye, according to certain embodiments.

FIG. 9C shows the ophthalmic surgical instrument of FIGS. 8A-8B inserted through a capsulotomy in the capsular bag of the eye shown in FIGS. 9A-9B, according to certain embodiments.

FIG. 10A shows the distal end of the ophthalmic surgical instrument shown in FIGS. 8A-8B with a tip having grooves, according to certain embodiments.

FIG. 10B shows the distal end of the ophthalmic surgical instrument shown in FIGS. 8A-8B with a tip having notches, according to certain embodiments.

FIG. 10C shows the distal end of the ophthalmic surgical instrument shown in FIGS. 8A-8B with a tip having wings, according to certain embodiments.

FIG. 10D shows the distal end of the ophthalmic surgical instrument shown in FIGS. 8A-8B with a tip having a textured surface, according to certain embodiments.

FIG. 11A shows a distal end of an ophthalmic surgical instrument with a tip, according to certain embodiments.

FIG. 11B shows the distal end of the ophthalmic surgical instrument with another tip, according to certain embodiments.

FIG. 12 illustrates a flowchart of a method for performing an ophthalmic surgical procedure, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

Current cataract surgeries often employ an ophthalmic surgical instrument (e.g., a phacoemulsification probe, a laser probe, or another suitable instrument) that is used to fragment a cataractous lens along with another ophthalmic surgical instrument (e.g., a secondary irrigation/aspiration (I/A) handpiece) for aspirating the fragmented lens and irrigating fluid (e.g., a balanced salt solution (BSS) or other irrigation solution) into the eye. As such, a surgeon may need to switch between the different ophthalmic surgical instruments one or more times throughout the surgery, as the surgeon can typically only hold two instruments. However, exchanging different instruments into and out of the eye throughout the surgery increases the risk of infection and the likelihood of unwanted and unintentional trauma to ocular tissues, and may also increase a duration of the surgery, which further increases risk to the patient.

Further, after the cataractous lens has been fragmented, a capsular wall of the lens may need to be polished to ensure sufficient removal of residual lens epithelial cells. However, due to a small surface area and rounded distal end, current ophthalmic surgical instruments struggle to remove the residual lens epithelial cells.

Accordingly, the embodiments described herein provide multifunctional instruments and a variety of tips that can be used with different ophthalmic surgical instruments, and which include features for the removal of residual lens epithelial cells. For example, when used with an ophthalmic surgical instrument, each of the tips herein enables the ophthalmic surgical instrument to be multifunctional, such that the ophthalmic surgical instrument can perform a certain surgical functionality (e.g., fragmentation, aspiration, and/or irrigation) while also being able to remove lens epithelial cells during capsule polishing. The multifunctional instruments described herein, therefore, reduce the need to switch between different instruments throughout the surgery, the likelihood of unwanted and unintentional trauma to ocular tissues, and the duration of the surgery.

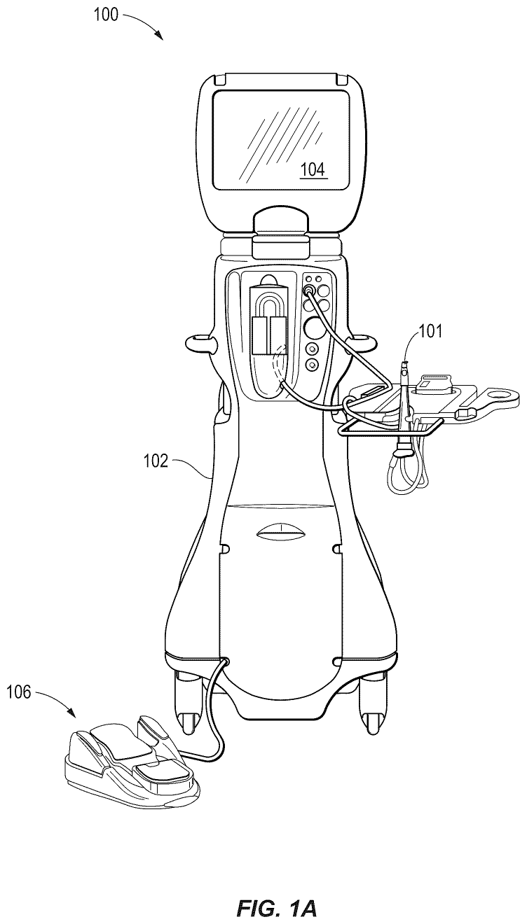

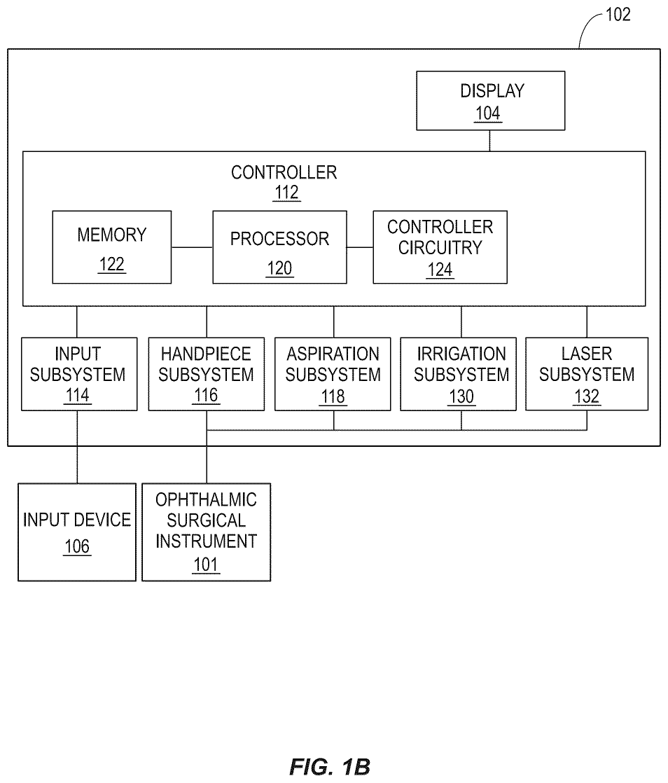

FIG. 1A shows an example ophthalmic surgical system 100 that may be used to perform ophthalmic procedures on an eye, according to certain embodiments. The ophthalmic surgical system 100 includes a console 102 (also referred to as a “surgical console”), which includes a display 104, an input device 106 (e.g., a foot pedal), and an ophthalmic surgical instrument 101 (also referred to as an “instrument” or a “handpiece”). The components of the ophthalmic surgical system 100 and the surgical console 102 are mechanically and/or electrically coupled as shown and described in more detail with reference to FIG. 1B.

The surgical console 102 may be similar to surgical consoles as shown and described in U.S. Pat. No. 9,931,447, the entire disclosure of which is hereby expressly incorporated herein by reference. The surgical console 102 may be similar to surgical consoles that have been known and used, such as the CENTURION® Vision System available from Alcon Laboratories, Inc. (Fort Worth, Texas) or the CONSTELLATION® Vision System available from Alcon Laboratories, Inc. (Fort Worth, Texas), or any other ophthalmic surgical console suitable for use with the principles described herein.

FIG. 1B shows example components of the surgical console 102 of the ophthalmic surgical system 100 shown in FIG. 1A, according to certain embodiments. As shown, the surgical console 102 includes a controller 112, an input subsystem 114, a handpiece subsystem 116, an aspiration subsystem 118, an irrigation subsystem 130, and a display 104. The controller 112 controls the operation of the surgical console 102 and is illustrated as being operationally coupled by a wired or wireless connection to the input device 106 via input subsystem 114, and to the ophthalmic surgical instrument 101 via handpiece subsystem 116, aspiration subsystem 118, irrigation subsystem 130, and a laser subsystem 132. The controller 112 includes a processor 120, a memory 122, and controller circuitry 124.

Processor 120 may be any type of general purpose processor or could be a processor specifically designed for driving the subsystems illustrated in FIG. 1B, such as an application-specific integrated circuit (“ASIC”). The processor 120 may be, or include, a microprocessor, a microcontroller, an embedded microcontroller, a programmable digital signal processor, or any other programmable device operable to execute instructions stored in the memory 122 for operating surgical console 102. For example, the processor 120 may execute instructions in the memory 122 to receive inputs provided by input device 106 through input subsystem 114 and, in response, send instructions to the aspiration subsystem 118 and/or the handpiece subsystem 116 for operating the ophthalmic surgical instrument 101. Further, the processor 120 may execute instructions to generate a user interface view for display by display 104. In some instances, the processor 120 may also be or include a programmable gate array, programmable array logic, or any other device of combinations of devices operable to process electric signals.

Memory 122 can be any type of storage device or non-transitory computer-readable medium, such as random-access memory (“RAM”) or read-only memory (“ROM”), which is operable to receive, store, or recall data, including, but not limited to, electronic, magnetic, or optical memory, whether volatile or non-volatile. The memory 122 stores instructions executed by the processor 120. In example embodiments, functionality disclosed herein can be provided by the processor 120 and the memory 122 (i.e., software based), by the controller circuitry 124 (i.e., hardware based), or by a combination thereof. The memory 122 may include code stored thereon. The code may include instructions that may be executable by the processor 120. The code may be created, for example, using any programming language, including but not limited to, C, C++, Java, Python, Rust, or any other programming language (including assembly languages, hardware description languages, and database programming languages). In some instances, the code may be a program that, when executed by the processor 120, causes the surgical console 102 to operate subsystems 114, 116, 118, 130 and/or 132 for, e.g., driving the ophthalmic surgical instrument 101 or other devices in communication with the surgical console 102.

Ophthalmic surgical instrument 101 may be any suitable ophthalmic surgical instrument that can be operated on the basis of the embodiments described herein. For example ophthalmic surgical instrument 101 may be a phacoemulsification (phaco) handpiece (also referred to as a “phaco probe”). Other examples of the ophthalmic surgical instrument 101 may include a laser probe instrument, an irrigation instrument, an aspiration instrument, or other similar probes.

Handpiece subsystem 116 is configured to facilitate the operation of the ophthalmic surgical instrument 101. For example, handpiece subsystem 116 may control the operations (e.g., activation and deactivation) of a component of the ophthalmic surgical instrument 101.

Aspiration subsystem 118 provides aspiration control for ophthalmic surgical instrument 101. In some embodiments, aspiration subsystem 118 may be operatively coupled to a surgical cassette during a surgical procedure. For example, the surgical cassette may be inserted into, attached to, and/or integrated with aspiration subsystem 118 via a coupling mechanism. When aspiration subsystem 118 is operatively coupled to a surgical cassette, aspiration subsystem 118 may control aspiration through the surgical cassette, which may in turn be coupled to the ophthalmic surgical instrument 101, for example, via a cable or other tether. In certain embodiments, the aspiration subsystem 118 includes one or more mechanical pumps having roller pump heads configured to engage with one or more corresponding pump assemblies on the surgical cassette. The engagement of the roller pump heads and pump assemblies generates a source of pressure and/or vacuum utilized during an ophthalmic surgical procedure.

Irrigation subsystem 130 provides irrigation control for ophthalmic surgical instrument 101. In some embodiments, irrigation subsystem 130 may be operatively coupled to a fluid bag during a surgical procedure. For example, the fluid bag may be inserted into, attached to, and/or integrated with irrigation subsystem 130 via a coupling mechanism. When irrigation subsystem 130 is operatively coupled to a fluid bag, irrigation subsystem 130 may control irrigation through the fluid bag, which may in turn be coupled to the ophthalmic surgical instrument 101, for example, via a cable or other tether. In certain embodiments, the irrigation subsystem 130 includes one or more mechanical plates configured to engage with the fluid bag. The engagement of the fluid bag generates a source of pressure utilized to irrigate fluid during an ophthalmic surgical procedure.

Laser subsystem 132 may comprise a laser that may be housed within the surgical console 102 or elsewhere, such as in a separate console that communicates with the surgical console 102. The laser subsystem 132 may include components for operating the laser, such as a power supply, controller, laser pumps, laser energy control, and/or monitor. In addition, the laser subsystem 132 may include components in the optical path of the laser output, such as one or more lenses, mirrors, and/or optical fibers.

In some embodiments, the laser subsystem 132 may be suitable for cataract surgery. In some embodiments, the output energy of the laser subsystem 132 is suitable for fragmentation and/or emulsification of a cataractous lens. In some examples, the laser output is used for fragmentation and/or emulsification of the lens to a sufficient degree for removal of the lens.

The laser may be any type of laser suitable for the desired application. The laser may output suitable electromagnetic radiation at any suitable wavelength. For example, the laser may emit electromagnetic radiation in one or more wavelengths in the visible, infrared, and/or ultraviolet wavelengths. The laser may operate or be operated to emit a continuous beam of electromagnetic radiation. Alternatively, the laser may operate or be operated to emit a pulsed beam.

In one example, the laser operates in the infrared range. For example, the laser may output electromagnetic radiation in the mid-infrared range, for example in a wavelength range of about 2.0 microns to about 4.0 microns. Some example wavelengths include about 2.5 microns to about 3.5 microns, such as about 2.775 microns, about 2.8 microns, or about 3.0 microns. Such a laser may be suitable, for example, for lens fragmentation or emulsification in cataract surgery, or for other procedures.

The laser subsystem 132 is designed to direct the laser electromagnetic radiation from the laser to an output port. The laser subsystem 132 may direct the laser electromagnetic radiation from the laser to the output port through one or more optical components, such as lenses and mirrors.

The ophthalmic surgical instrument 101 may be optically connected to the laser subsystem 132 to receive the laser electromagnetic radiation from the output port. The ophthalmic surgical instrument 101 may be connected to the laser subsystem 132, e.g., by a cable with an optical fiber. The connection (e.g., cable) may be flexible and relatively long to give the operator flexibility in maneuvering the ophthalmic surgical instrument 101 at some distance away from the laser subsystem 132. The laser electromagnetic radiation may be transmitted to the ophthalmic surgical instrument 101 from the laser subsystem 132 through the optical fiber of the connecting cable. The optical fiber of the connecting cable may continue through the ophthalmic surgical instrument 101 or may be optically connected to an optical fiber in the ophthalmic surgical instrument 101. In either case, the ophthalmic surgical instrument 101 may include an optical fiber that terminates at the distal end of the ophthalmic surgical instrument 101. The optical fiber may carry the laser electromagnetic energy from the laser and emit it from the distal end of the optical fiber at the distal end of the ophthalmic surgical instrument 101 to the desired target, such as a lens or lens fragment in the eye of a patient.

Input device 106 may be any device that is capable of receiving commands from the user of the surgical console 102 in order to operate the ophthalmic surgical instrument 101 and/or other components of the surgical console 102. In FIG. 1A, input device 106 is illustrated as a foot pedal, however, other types of input devices are also within the scope of the disclosure. In one example, the user provides a command to the input device 106, which is received and relayed to the controller 112 by the input subsystem 114. In response, the controller 112 sends instructions to the handpiece subsystem 116, aspiration subsystem 118, and/or irrigation subsystem 130 to control the operations of the ophthalmic surgical instrument 101 based on the user command.

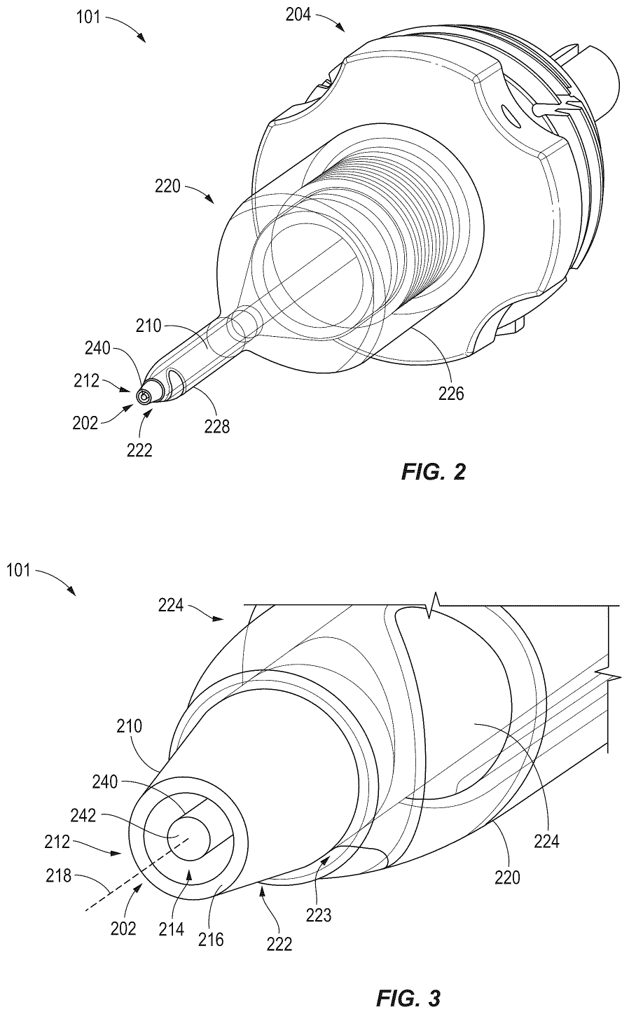

FIG. 2 shows an example of the ophthalmic surgical instrument 101 with a laser optical fiber 240 (e.g., a sapphire optical fiber), according to certain embodiments. FIG. 3 shows an enlarged view of the distal end 202 of the ophthalmic surgical instrument 101 of FIG. 2, according to certain embodiments. Accordingly, FIGS. 2 and 3 are described together herein for clarity purposes.

The ophthalmic surgical instrument 101 comprises a housing 204 and a shaft 210, e.g., a cannula, that extends from the distal end 212 of the housing 204. The housing 204 may be hollow with an internal chamber in which internal components of the instrument 101 may be housed and protected. The housing 204 may have an external surface that can be grasped by an operator of the instrument 101, such as a surgeon.

The shaft 210 may be tubular, e.g., a hollow tube such as a cannula, that has an aspiration port 214 at its distal end 212 which may be used for aspiration. For example, by applying suction through an aspiration channel from the proximal end of an aspiration luer at the proximal end of the housing 204, fluid and/or tissue, such as lens or other tissue fragments, may be aspirated through the aspiration port 214 in the distal end 212 of the shaft 210.

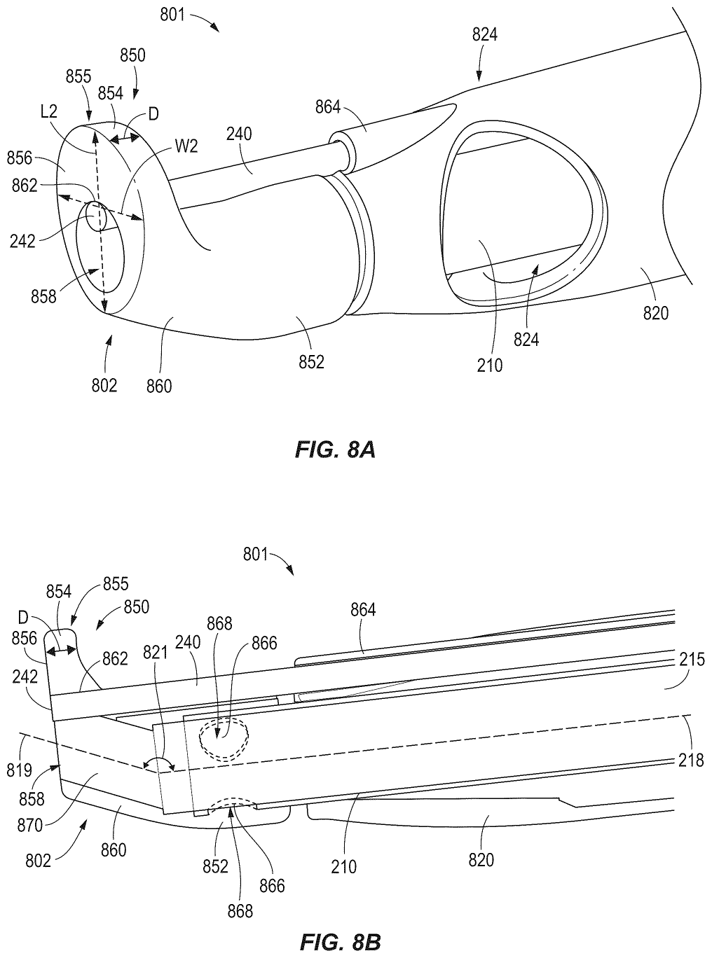

The optical fiber 240 may be located inside of the shaft 210, as shown. The optical fiber tip 242 may be flush (in the same plane as) the end face 216 of the shaft 210. In other embodiments, the optical fiber tip 242 may extend beyond the end face 216 of the shaft 210, or the end face 216 of the shaft 210 may extend beyond the optical fiber tip 242. The optical fiber tip 242 does not need to be inside of the aspiration port 214 but only in close proximity so that the material affected by the laser action can be readily aspirated. Examples of the optical fiber 240 located outside of the shaft 210 are shown in FIGS. 8A-8B, 9A-9B, and 10A-10D.

The ophthalmic surgical instrument 101 may also include an irrigation sleeve 220. The irrigation sleeve 220 may serve to direct an irrigation fluid (e.g., saline) to the distal end 202 of the instrument 101. The housing 204 may have an irrigation supply line through which an irrigation fluid may be introduced. The irrigation sleeve 220 may be coupled directly to the housing 204 or coupled to the housing 204 through another part of the ophthalmic surgical instrument 101. In one example, the housing 204 may have external threads at its distal end, and the irrigation sleeve 220 may have internal threads at its proximal end, by which the irrigation sleeve 220 may be attached to the housing 204. As can be seen in FIG. 2, the irrigation sleeve 220 generally has a proximal hub 226 having a relatively larger diameter for coupling to the housing 204 and a distal tube 228 in the shape of a narrow tube having a relatively smaller diameter for fitting around the shaft 210 at a small enough dimension for inserting through an incision in an eye.

In the illustrated example, the irrigation sleeve 220 is positioned around the shaft 210 to provide a fluid passageway or channel from the position of its attachment to the housing 204 through the space between the irrigation sleeve 220 and the shaft 210. The irrigation sleeve 220 has an end opening 223 at its distal end 222 through which the distal end 212 of the shaft 210 extends and one or more side openings 224 at its distal end 222 adjacent to the end opening 223. At the end opening 223, the irrigation sleeve 220 has a snug fit around the shaft 210. When an irrigation fluid is introduced through the supply line in the housing 204, it passes through the channel of the irrigation sleeve 220 and out of the side opening(s) 224 at the distal end 222 of the irrigation sleeve 220.

As an example, the distal tube 228 of the irrigation sleeve 220 may have an outer diameter in a range of 0.030 inches to 0.080 inches to fit through an incision in an eye and an inner diameter in a range of 0.020 inches to 0.070 inches to accommodate the shaft 210. The end opening 223 at the distal end 222 of the irrigation sleeve 220 through which the distal end 212 of the shaft 210 projects may be a circular opening having a diameter in a range of 0.010 inches to 0.065 inches, which may be equal to or slightly smaller than the outer diameter of the distal end 212 of the shaft 210 to form a snug fit around the distal end 212 of the shaft 210. The examples of dimensions and ranges of dimensions represent possible embodiments; other embodiments with different dimensions may be used.

The irrigation sleeve 220 may be made of an elastomeric material such as a compliant silicone rubber. The irrigation sleeve 220 alternatively may be made of other materials, such as polyurethane, ethylene propylene, neoprene, or other suitable materials. An elastomeric material for the irrigation sleeve 220 allows some compliance and facilitates the snug fit between irrigation sleeve 220 and the shaft 210 at the area of the end opening 223. An elastomeric material for the irrigation sleeve 220 also facilitates a seal or snug fit between the irrigation sleeve 220 and the adjacent eye tissue at the incision site, such as the cornea or sclera, which can help minimize leakage from the eye between the eye tissue and the irrigation sleeve 220.

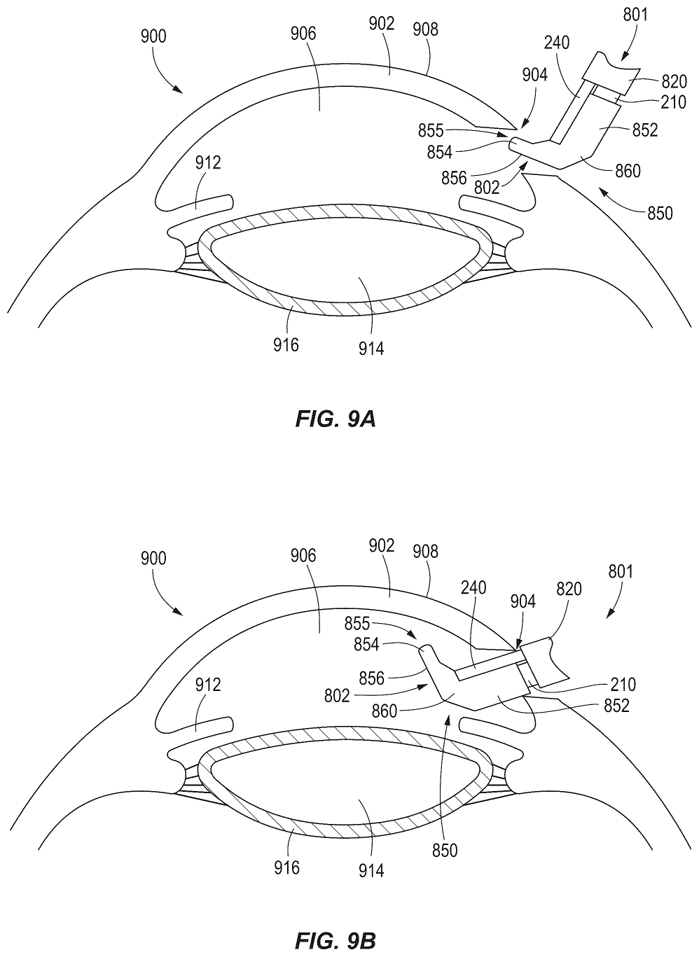

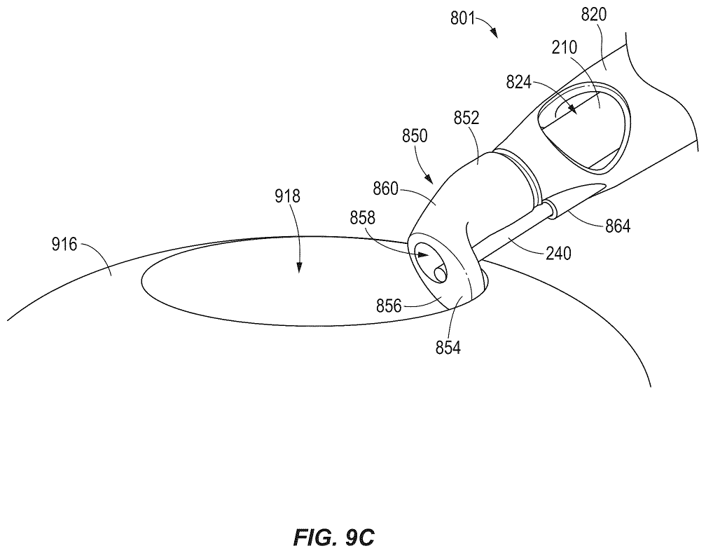

In the operation of the ophthalmic surgical instrument 101, the operator inserts the distal end 202 of the ophthalmic surgical instrument 101 through a suitable incision in a patient's eye (e.g., as shown in FIGS. 9A-9B) and positions the distal end 202 of the ophthalmic surgical instrument 101 at a desired location, such as adjacent to a cataractous lens of a patient. The distal end 212 of the shaft 210 is directed toward a treatment area, with the tip 242 at the distal end of the optical fiber 240 positioned facing the treatment area. Irrigation fluid such as saline may be supplied from the surgical console through the irrigation supply line and irrigation sleeve 220, such that it flows out of the side opening(s) 224 to the target area. The laser subsystem may be activated to emit laser energy from the tip 242 of the optical fiber 240 (i.e., the distal end of the optical fiber 240), which can break up and emulsify the desired tissue, such as the cataractous lens. At the same time, a pumping module may be used to apply suction through the shaft 210, thereby suctioning through the aspiration port 214 fluid and tissue and/or lens fragments that have been separated by the action of the laser.

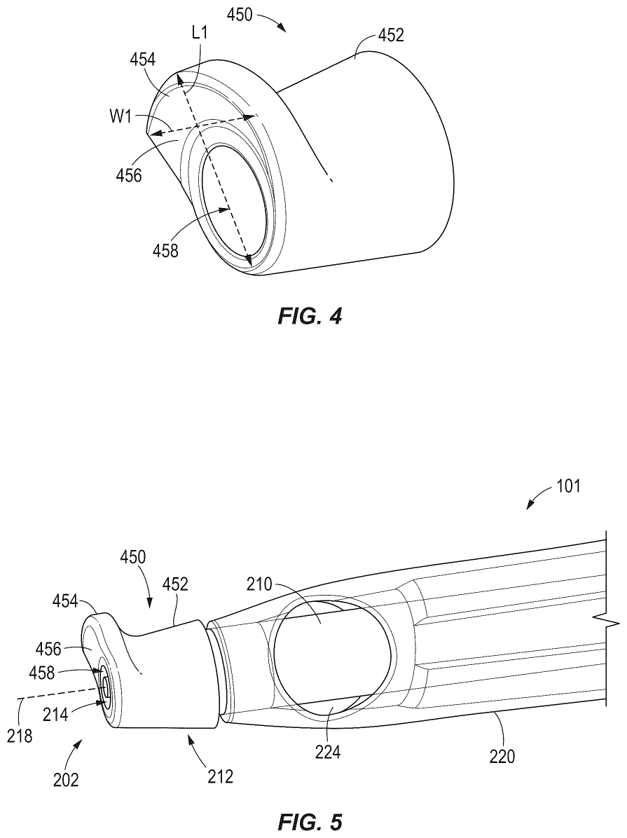

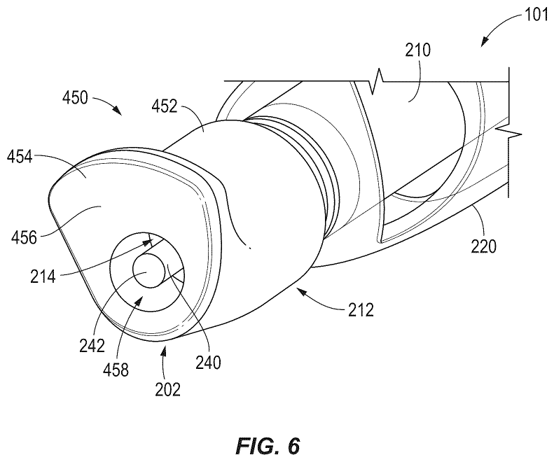

FIG. 4 shows a tip 450 for the ophthalmic surgical instrument 101, according to certain embodiments. FIG. 5 shows the distal end 202 of the ophthalmic surgical instrument 101 with the tip 450 of FIG. 4, according to certain embodiments. FIG. 6 shows another view of the distal end 202 of the ophthalmic surgical instrument 101 of FIG. 5, according to certain embodiments. Accordingly, FIGS. 4, 5, and 6 are described together herein for clarity purposes.

The tip 450 has a cylindrical hub 452 and a projection 454. In some embodiments, the projection may be one of the projections described in U.S. patent application Ser. No. 18/451,211 titled “Devices and Methods for Improved Followability in Laser-Based Ocular Procedures”, filed on Aug. 17, 2023, whose inventors are Francisco Javier Ochoa and John Morgan Bourne, which is hereby incorporated by reference in its entirety as though fully and completely set forth herein.

The cylindrical hub 452 is configured to fit over and on the distal end 212 of the shaft 210, as can be seen in FIGS. 5 and 6. The tip 450 has a center opening 458 that provides an entranceway to the aspiration port 214 (e.g., see FIG. 3) of the shaft 210 for aspiration. The optical fiber 240 is located inside of the shaft 210 and inside of the center opening 458 of the tip 450. In an alternative example, the tip 450 has a center opening 458 that provides an entranceway to the aspiration port 214 of the shaft 210 for aspiration, but the optical fiber 240 is located outside of the shaft 210. For example, in such an embodiment, the optical fiber 240 may be located either inside or outside of the center opening 458 of the tip 450. Locating the optical fiber 240 outside of the shaft 210 can help reduce clogging of the aspiration channel, by having the laser action offset from the aspiration channel.

When the tip 450 is coupled to the instrument 101 as shown in FIGS. 4, 5, and 6, the projection 454 is located at the distal end 212 of the shaft 210 and at the distal end 202 of the instrument 101. The projection 454 has a distal surface 456 in proximity to the distal tip 242 of the optical fiber 240. Thus, the distal surface 456 of the projection 454 faces the treatment area when the optical fiber tip 242 faces the treatment area.

When the tip 450 is in use, the distal surface 456 provides instrument 101 a surface area facing the treatment area that is significantly larger than when the tip is not in use. For example, as shown in FIG. 3, in the illustrated instrument 101 without the projection 454, the surface area of instrument 101 facing the treatment area would be limited to the end face 216 of the shaft 210, aside from the tip 242 of the optical fiber 240 itself. Therefore, the projection 454 provides a significantly larger surface area facing the treatment area as compared to the end face 216 of the shaft 210.

In one example, the end face 216 has an outer diameter of 0.0243 inches and an inner diameter of 0.0187 inches, with a surface area of 0.000198 square inches. In one example of the projection 454, the surface area of the distal surface 456 facing the treatment area is 0.00164 square inches. Thus, in this example, the surface area of the distal surface 456 of the projection 454 is more than eight times greater than the surface area of the end face 216 of the shaft 210.

In other examples, a surface area of the distal surface 456 may be at least two times greater than a surface area of the end face 216 of the shaft 210, or at least four times greater than a surface area of the end face 216 of the shaft 210, or at least six times greater than a surface area of the end face 216 of the shaft 210. The distal surface 456 of the projection 454 may have a length (L1) of 0.020 inches to 0.090 inches and a width (W1) of 0.020 inches to 0.060 inches. Smaller or larger lengths and/or widths may be used. In one example, the distal surface 456 of the projection 454 has a length of about 0.050 inches and a width of about 0.020 inches. The distal surface 456 of the projection 454 may be dished or curved in whole or in part, thereby presenting a concave surface facing the treatment area, in better conformity to the shape of the bubbles induced by the laser action.

The projection 454, with its distal surface 456 presenting a surface area that faces the area treated by the laser, helps inhibit the bubbles formed by the laser from moving away from the optical fiber tip 242. Without the surface area of the projection 454, the action of the laser and the bubble dynamics can, in certain situations, cause the bubbles formed by the laser to move away from the distal end of the instrument. This can reduce the efficiency of the laser action or otherwise detrimentally affect the performance of the instrument 101.

Without a projection and associated surface area facing the treatment area, a system may have good holding power for high vacuum levels or low power settings, but at higher laser power settings the laser action and bubble dynamics may cause repulsion of the resultant bubbles. Accordingly, the cutting power or emulsification efficiency decreases significantly, since the target tissue or material is being moved away from the distal end of the instrument tip as opposed to broken up and aspirated into the cannula.

The use of a projection with a surface area facing the treatment area (e.g., surface area of distal surface 456) helps inhibit the distal movement of the bubble action from the optical fiber tip 242. At a minimum, it reduces the repulsive effects of the laser-induced bubble dynamics. In some embodiments, it can reverse the effect such that repulsion becomes attraction. In cataract surgical terms, the use of a projection with its surface area facing the treatment area improves followability.

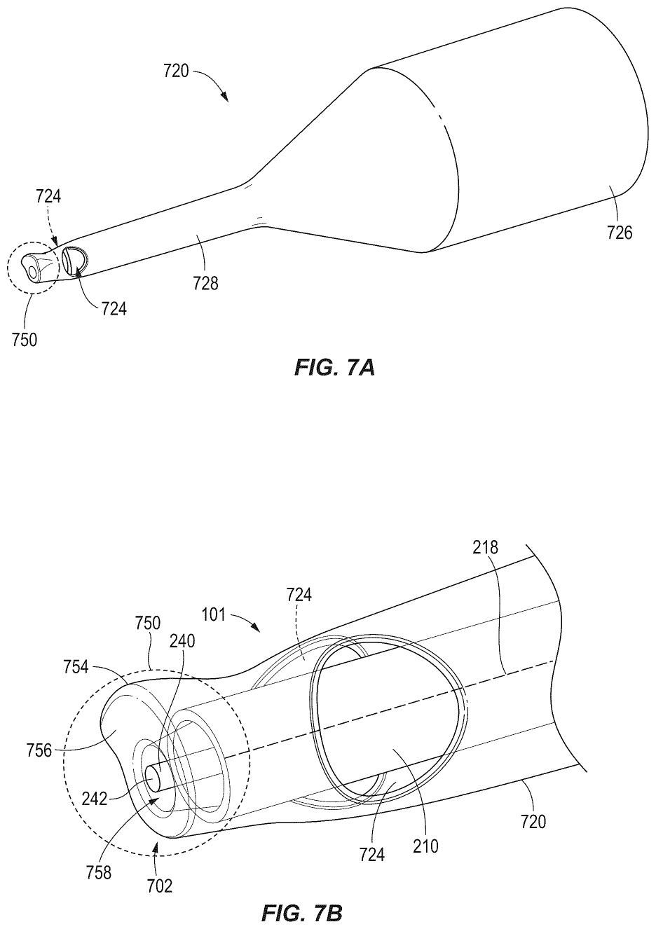

In some embodiments, the tip 450 with the projection 454 is a plastic part that is added to the shaft 210 (e.g., a metal cannula). The projection 454 can also be shaped out of the shaft or cannula itself or integrated as part of the irrigation sleeve 220 (e.g., as shown in FIGS. 7A-7B). The shape of the projection 454 can have different configurations depending on the performance requirements.

In some embodiments, the tip 450, including the hub 452 and the projection 454, may be transparent, in order to facilitate visualization by the operator through the material. The tip 450 may be comprised of a polymeric material. As an example, the tip 450 may be comprised of polycarbonate.

In some embodiments, the projection 454 and the distal surface 456 may be asymmetrical about a plane along the longitudinal axis 218 of the shaft 210. This asymmetry can allow a cutting or shaving action on one side of the instrument 101.

In some embodiments, the projection 454 may be angled with respect to the longitudinal axis 218 of the shaft 210 (e.g., at an angle from 0° (degrees) to 90° (perpendicular) relative to the longitudinal axis 218). This can help facilitate insertion of the distal end 202 of the instrument 101 into the eye (e.g., as shown in FIGS. 9A-9B). That is, the operator can tilt the instrument 101 at a first angle to maneuver the projection 454 into the incision in the eye (e.g., as shown in FIG. 9A), and then tilt the instrument 101 at a second angle to maneuver the shaft 210 through the incision in the eye (e.g., as shown in FIG. 9B).

FIG. 7A shows an irrigation sleeve 720 for the ophthalmic surgical instrument 101 shown in FIG. 2, according to certain embodiments. FIG. 7B shows the ophthalmic surgical instrument 101 and the sleeve 720 shown in FIG. 7A coupled together, according to certain embodiments. Accordingly, FIGS. 7A-7B are described together herein for clarity purposes.

The irrigation sleeve 720 may be the same as the irrigation sleeve 220 described with reference to FIG. 2, except that the sleeve 720 includes a tip 750 that incorporates a projection 754 with a distal surface 756 (instead of the tip 450 with the projection 454 and the distal surface 456). In such embodiments, the projection 754 is part of the irrigation sleeve 720 and may be molded as part of its distal end 702. The distal surface 756 functions similarly to the distal surface 456 of the tip 450.

In this illustrated example, the irrigation sleeve 720 has a center opening 758 that provides an entranceway to the channel of the shaft 210 for aspiration and side openings 724 for fluid irrigation. Additionally, the optical fiber 240 is located inside of the shaft 210 and the center opening 758. In an alternative example, the irrigation sleeve 720 again has a center opening that provides an entranceway to the channel of the shaft 210 for aspiration, but the optical fiber 240 is located outside of the shaft 210, and the optical fiber 240 may be located either inside or outside of the center opening of the irrigation sleeve 720.

The irrigation sleeve 720, including the projection 754, may be transparent, in order to facilitate visualization by the operator through the material. The projection 754 may be formed of the same material or a different material as the irrigation sleeve 220, e.g., a polymeric material such as polycarbonate or an elastomeric material such as silicone rubber.

The projection 754 and the distal surface 756 may be asymmetrical about a plane along the longitudinal axis 218 of the shaft 210. This can allow a cutting or shaving action on one side of the instrument 101. Also, the projection 754 may be angled with respect to the longitudinal axis 218 of the shaft 210 (e.g., at an angle from 0° to 90° (perpendicular) relative to the longitudinal axis 218), to facilitate insertion of the distal end 702 of the instrument 101 into the eye, as described above.

The irrigation sleeve 720 may be coupled directly to the housing 204 or coupled to the housing 204 through another part of the ophthalmic surgical instrument 101. In one example, the housing 204 may have external threads at its distal end, and the irrigation sleeve 720 may have internal threads at its proximal end, by which the irrigation sleeve 720 may be attached to the housing 204. In some examples, the irrigation sleeve 720 may be slideable relative to the housing 204, such that the optical fiber 240 may be exposed and/or covered by moving the irrigation sleeve 720 longitudinally (e.g., along the longitudinal axis 218) relative to the shaft 210.

As can be seen in FIG. 7A, the irrigation sleeve 720 has a proximal hub 726 configured to be coupled to the housing 204 and a distal tube 728 having a smaller diameter (relative to the diameter of the proximal hub 726) for fitting around the shaft 210. The sleeve 720 with projection 754 may be coupled to a commercially available phacoemulsification tip (e.g., a tip without a projection such as a balanced phacoemulsification tip or a hybrid phacoemulsification tip) to safely reach residual cortex under a capsulotomy with the projection 754.

FIG. 8A shows a distal end of an ophthalmic surgical instrument 801 with the optical fiber 240 disposed outside the shaft 210, according to certain embodiments. FIG. 8B is a cross-sectional side view of the ophthalmic surgical instrument 801 shown in FIG. 8A, according to certain embodiments. Accordingly, FIGS. 8A-8B are described together herein for clarity purposes.

In FIGS. 8A-8B, the optical fiber 240 extends distally from a fiber tube 864 disposed adjacent to the shaft 210, and through a tip 850 coupled to the shaft 210. In particular, the optical fiber 240 is located outside of the shaft 210 and inside of a center opening 858 of the tip 850. The fiber tube 864 may be laser welded to the shaft 210 and is configured to help prevent the optical fiber 240 from breaking. In the illustrated example, an irrigation sleeve 820, which is similar to the irrigation sleeve 220, is positioned around the shaft 210 and the fiber tube 864. The sleeve 820 includes two side openings 824 for irrigating fluid (e.g., BSS).

FIGS. 8A-8B illustrate a tip 850 including a substantially flat distal surface 856, a body 860 between cylindrical hub 852 and projection 854, and a slot 862 disposed through the projection 854. The tip 850 also includes a plurality of inner protrusions or projections 866 that are circumferentially arranged on the inner surface of the cylindrical hub 852 and extend inwards from the cylindrical hub 852 toward the longitudinal axis thereof. The inner projections 866 are configured to interact with or be positioned into corresponding apertures 868 similarly arranged on the outer surface of the distal end of the shaft 210. By fitting into the corresponding apertures 868, the inner projections 866 are configured to removably couple the tip 850 to the shaft 210.

The projection 854 of the tip 850 has a thin cross-sectional profile (D) at its edge 855. The thin cross-sectional profile (D) of the projection 854 may have a depth of 0.005 inches to 0.10 inches. The thin cross-sectional profile (D) of the projection 854 helps facilitate easier insertion of the ophthalmic surgical instrument 801 into the eye. Additionally, the projection 854 can be used as a hook to gently pull on a capsulotomy to further extend the reach of the ophthalmic surgical instrument 801. For example, the projection 854 may be used to reach under the capsulotomy to facilitate removal of cortical material, including sub-incisional cortex, which may be particularly difficult to reach. Such uses of the ophthalmic surgical instrument 801 with the tip 850 are described in further detail with reference to FIGS. 9A-9C.

The flat distal surface 856 of the tip 850 helps provide a lens capsule-friendly surface, which can be used to operate on and around the capsule membrane. In one example of the projection 854, the surface area of the distal surface 856 facing the treatment area is 0.00164 square inches, which is more than eight times greater than the surface area of the end face 216 of the shaft 210. In other examples, a surface area of the distal surface 856 of the projection 854 may be at least two times greater than a surface area of the end face 216 of the shaft 210, or at least four times greater than a surface area of the end face 216 of the shaft 210, or at least six times greater than a surface area of the end face 216 of the shaft 210. The distal surface 856 of the projection 854 may have a length (L2) of 0.020 inches to 0.090 inches and a width (W2) of 0.020 inches to 0.060 inches. Smaller or larger lengths and/or widths may be used. In one example, the distal surface 856 of the projection 854 has a length of about 0.050 inches and a width of about 0.020 inches.

The optical fiber 240 is disposed through the slot 862 and is substantially flush with the distal surface 856. In the illustrated example, the optical fiber 240 is positioned along an edge of the center opening 858. By positioning the optical fiber 240 near an outer portion of the center opening 858, flow of tissue and/or other material aspirated by the ophthalmic surgical instrument 801 through the center opening 858 is improved in comparison to the flow of materials through the center opening 458. Accordingly, the positioning of the optical fiber 240 as shown in FIGS. 8A-8B helps reduce clogging of the aspiration channel. Further, when the optical fiber tip 242 is flush with the distal surface 856, the optical fiber 240 is less likely to break during use of the ophthalmic surgical instrument 801.

The body 860 of the tip 850 defines an aspiration channel 870 therein which fluidly connects the center opening 858 to channel 215 of the shaft 210. In the illustrated example, a longitudinal axis 819 of the body 860 defines an angle 821 relative to the longitudinal axis 218 of the shaft 210. The angle 821 defined by the longitudinal axes 218, 819 is between, for example, 95° and 175° (e.g., between 100° and 170°, 105° and 165°, 110° and 160°, or 115° and 155°). By angling the channel 870 of the tip 850 relative to the channel 215 of the shaft 210, tissue and/or other materials aspirated by the ophthalmic surgical instrument 801 are guided away from the optical fiber 240 and are therefore less likely to get caught on and/or break the optical fiber 240.

In some embodiments, the tip 850, including the hub 852 and the projection 854, may be transparent, in order to facilitate visualization by the operator through the material. The tip 850 may be comprised of a polymeric material. As an example, the tip 850 may be comprised of polycarbonate. In some embodiments, the projection 854 can also be shaped out of the shaft or cannula itself, such that the projection 854 may be formed of the same material as the shaft or cannula. The shape of the projection 854 can have different configurations depending on the performance requirements.

Although the ophthalmic surgical instrument 801 is shown as including the optical fiber 240 with the optical fiber tip 242 extending to the distal surface 856 of the projection 854, in some embodiments, the optical fiber tip 242 may not extend to the distal surface 856, and in some other embodiments, the ophthalmic surgical instrument 801 may not include the optical fiber 240 (e.g., in an ultrasonic phacoemulsification tip).

FIGS. 9A-9B show the ophthalmic surgical instrument 801 of FIGS. 8A-8B being inserted into an eye 900, according to certain embodiments. For brevity, reference numbers for the eye 900 are only shown for cornea 902, outer surface 908 of the cornea 902, an anterior segment 906 (e.g., an anterior chamber), an iris 912, a lens 914, and a capsular bag 916.

In FIG. 9A, the ophthalmic surgical instrument 801 is introduced into the eye 900 via a clear corneal incision, such as incision 904. The incision 904 is configured to provide the ophthalmic surgical instrument 801 access to the anterior segment 906 of the eye 900. To help with the insertion of the ophthalmic surgical instrument 801, the ophthalmic instrument 801 is angled such that the projection 854 is first inserted into the incision 904. The projection 854 facilitates easier insertion of the ophthalmic surgical instrument 801 into the eye 900 due to its thin profile and rounded edge 855, which is less likely to bulge into and/or cause unwanted cutting at surrounding areas of the outer surface 908 of the cornea 902 near the incision 904.

Once the ophthalmic surgical instrument 801 is introduced into the eye 900 as shown in FIG. 9B, the ophthalmic surgical instrument 801 can be maneuvered in the anterior segment 906 of the eye 900 to interact with the capsular bag 916 of the lens 914. For example, during cataract surgery, the ophthalmic surgical instrument 801 can be used to fragment the lens 914, and to aspirate the fragments out of the eye 900. Throughout the procedure, fluid can be irrigated into the eye 900 through the sleeve 820.

FIG. 9C shows the ophthalmic surgical instrument 801 of FIGS. 8A-8B inserted through a capsulotomy 918 in the capsular bag 916 of the eye 900, according to certain embodiments. In some embodiments, a laser is used to create the capsulotomy 918 before the ophthalmic surgical instrument 801 is inserted therethrough. Once the tip 850 is inserted inside the capsular bag 916, the projection 854 may be used to help facilitate cortex removal (e.g., using the same laser or ultrasonic handpiece tip used for the capsulotomy 918). In addition, residual lens epithelial cells may be removed by simultaneously aspirating while moving the tip 850 around inside of the capsular bag 916, and along an inner wall of the capsular bag 916. The projection 854 can also be used to reach under an edge of the capsulotomy 918 for a more thorough polishing inside the capsular bag 916.

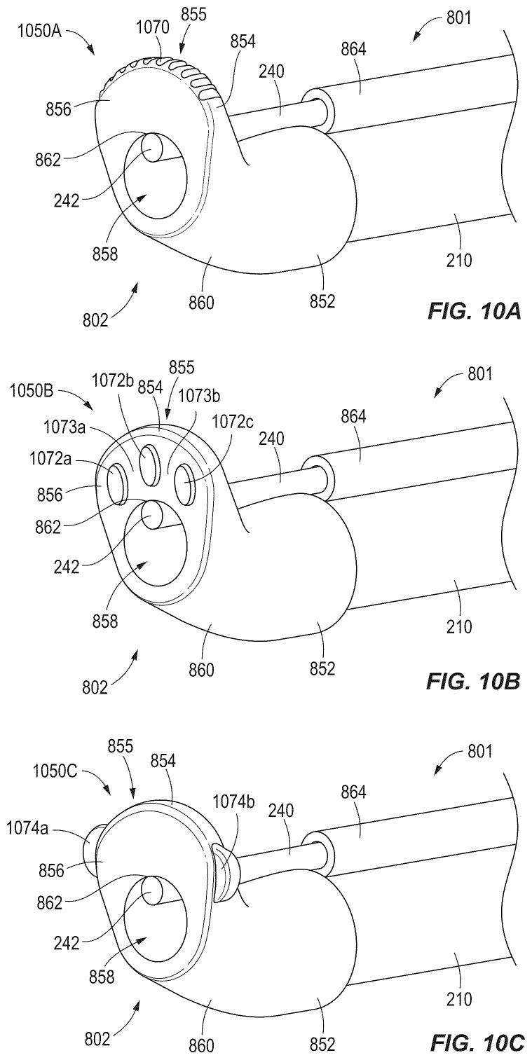

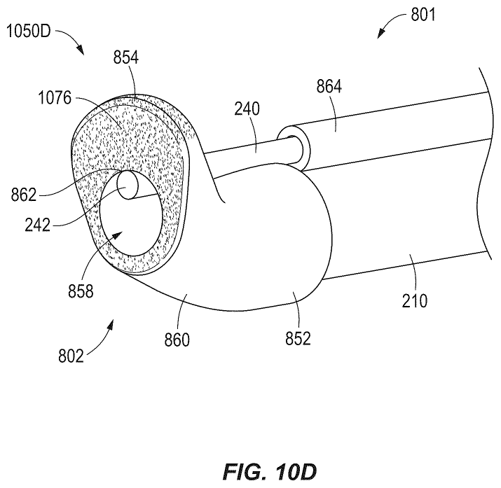

In some embodiments, for capsule polishing, the tip 850 may also comprise additional features and/or surface texture to facilitate removal of residual lens epithelial cells and sub-incisional cortex. Examples of tips with the additional features and/or the surface texture are described with reference to FIGS. 10A-10D.

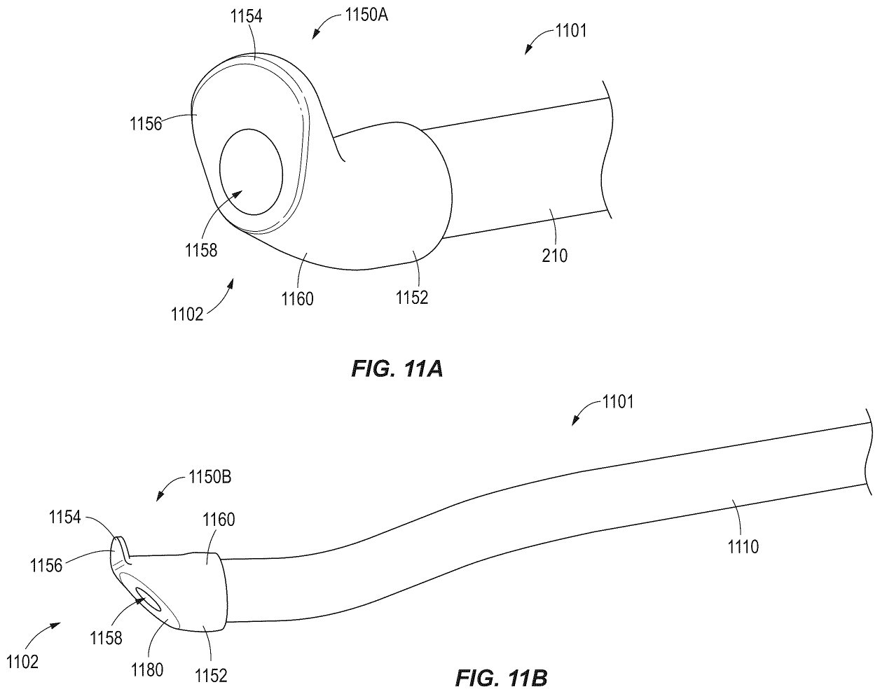

Note that one or more of the ophthalmic surgical instruments in FIGS. 10A-10D and 11A-11B are illustrated in a genericized manner (i.e., without additional components). For example, a sleeve is typically used with one or more of the ophthalmic surgical instruments in FIGS. 10A-10D and 11A-11B (to irrigate fluid into the eye 900); however, the sleeve and one or more other components have been omitted for visual clarity.

FIG. 10A shows the distal end 802 of the ophthalmic surgical instrument 801 with a tip 1050A having grooves 1070, according to certain embodiments. The grooves 1070 are located on the edge 855 of the projection 854. The grooves 1070 are formed by a plurality of ridges and depressions that are equally disposed across the edge 855 of the projection 854. As an example, each of the plurality of ridges and depressions of the grooves 1070 extend across the edge 855 by 0.002 inches to 0.020 inches. While the grooves 1070 are shown on the edge 855 of the projection 854, alternatively or additionally, they may be placed on other areas of the projection, including the distal surface 856. Additionally, in some embodiments, the grooves may extend further or completely around the outer edge of the distal surface 856.

FIG. 10B shows the distal end 802 of the ophthalmic surgical instrument 801 with a tip 1050B having notches labeled 1072 (1072a, 1072b, and 1072c), according to certain embodiments. The notches 1072 are located on the distal surface 856 of the projection 854. The tip 1050B also includes one or more sub-projections labeled 1073 (1073a and 1073b), which form the one or more notches 1072 therebetween. As an example, each of the notches 1072 extend outward from the distal surface 856 by 0.002 inches to 0.020 inches. While the notches 1072 are shown as having an oval shape, other shapes may also be used (e.g., rectangular, triangular, etc.). Additionally, while three notches 1072 are shown in FIG. 10B, in other embodiments, other numbers of notches (and corresponding sub-projections therebetween) may be used. For example, 1, 2, 4, 5, 10, 15, 20, etc. notches 1072 may be used.

FIG. 10C shows the distal end 802 of the ophthalmic surgical instrument 801 with a tip 1050C having wings labeled 1074 (1074a and 1074b), according to certain embodiments. The wings 1074 are located on the projection 854 and extend laterally outwards therefrom. As an example, the wings 1074 extend outward from the projection 854 by 0.005 inches to 0.020 inches. In the illustrated example, the wings 1074 are semi-circular, but may also be rectangular, triangular, or other similar shape. Further, while the embodiment shown in FIG. 10 shows two wings, fewer or more wings can be used (e.g., 1, 3, 4, 5, 10, etc.). The wings 1074 may also be disposed at different locations on the projection 854.

FIG. 10D shows the distal end 802 of the ophthalmic surgical instrument 801 with a tip 1050D having a distal surface 1076 that is textured, the textured distal surface 1076 including a portion of the projection 854, according to certain embodiments. The textured distal surface 1076 has a roughened surface texture which may be etched (e.g., with a laser), abraded (e.g., with sandpaper), and/or coated (e.g., a tacky coating) onto the surface. As an example, the roughened surface texture of the distal surface 1076 has a roughness with an Ra between 0.012 micrometers (μm) to 25 micrometers.

Note that the features (e.g., grooves 1070, notches 1072, wings 1074, and textured surface 1076) shown in FIGS. 10A-10D are each shown on different tips, but that one tip may include any combination of two or more of such features. For example, a tip may include the grooves 1070 and the notches 1072, the notches 1072 and the wings 1074, or the wings 1074 and the textured surface 1076. Further, the features described with reference to FIGS. 10A-10D may also be implemented with the projection 754 of the sleeve 720 shown in FIGS. 7A-7B.

The grooves 1070, the notches 1072, the wings 1074, and the textured surface 1076 shown in FIGS. 10A-10D each provide additional scraping edges for cortical material removal (especially under the surface of a capsulotomy). For example, during capsule polishing, these features help with the removal of residual lens epithelial cells when the tip (e.g., tip 1050A-D) is slid against a capsule wall. The residual lens epithelial cells removed from the capsule wall are then aspirated through the center opening 858.

FIG. 11A shows a distal end 1102 of an ophthalmic surgical instrument 1101 with a tip 1150A, according to certain embodiments. The tip 1150A is a phacoemulsification tip configured for ultrasonic removal of lens material without an optical fiber. The tip 1150A includes cylindrical hub 1152, projection 1154, distal surface 1156, center opening 1158, and body 1160 similar to the tip 850 shown in FIGS. 8A-8B but does not include the slot 862 for the optical fiber 240.

FIG. 11B shows the distal end 1102 of the ophthalmic surgical instrument 1101 with another tip 1150B, according to certain embodiments. The tip 1150B is another example of a phacoemulsification tip without the optical fiber 240, which may be used on a hybrid phacoemulsification needle. An example hybrid phacoemulsification needle is described in U.S. Pat. No. 11,185,442 titled “Hybrid Phacoemulsification Needle” filed May 8, 2019 which is hereby incorporated by reference in its entirety as though fully and completely set forth herein.

The tip 1150B includes an angled distal surface 1180 around the center opening 1158 with the projection 1154 and the flat distal surface 1156 extending outwards therefrom. Additionally, in the illustrated example, the ophthalmic surgical instrument 1101 includes a shaft 1110 having a curved or bent portion along a length thereof; however, in some other embodiments, the shaft 1110 may also be substantially straight (without the curved portion).

In one example of the hybrid phacoemulsification needle with the tip 1150B, the shaft 1110 has an interior surface, an exterior surface, and a distal end terminating in a distal edge. The shaft 1110 further has a central bore extending therethrough. The central bore is defined by the interior surface of the hollow shaft 1110; and the tip 1150B may include a first over mold located on the exterior surface and distal edge of the hollow shaft 1110, the first over mold covering at least a portion of a periphery of the exterior surface of the hollow shaft 1110, the first over mold covering the distal edge and terminating at the central bore.

In some other examples of the hybrid phacoemulsification needle with tip 1150B, the shaft 1110 comprises a through hole extending from the exterior surface to the interior surface of the shaft 1110, where the first over mold substantially fills the through hole. The first over mold may have a rounded front edge located over the distal edge of the hollow shaft 1110 and a rounded trailing edge located on the exterior surface of the hollow shaft 1110. The first over mold may extend circumferentially around the entire perimeter of the exterior surface of the hollow shaft 1110. The first over mold may be made of a polymer.

In yet some other examples of the hybrid phacoemulsification needle with tip 1150B, the phacoemulsification needle further comprises a second over mold located on the first over mold, the second over mold covering at least a portion of a periphery of an exterior surface of the first over mold, the second over mold covering the rounded front edge of the first over mold and terminating at the central bore. The second over mold may be embedded in the first over mold such that the exterior surface of the first over mold is continuous with an exterior surface of the second over mold. The second over mold may extend circumferentially around the entire perimeter of the exterior surface of the hollow shaft. The second over mold may be made of silicone.

In various embodiments, the projections (e.g., projections 454, 854, and 1154) and their various features (e.g., grooves 1070, notches 1072, and/or wings 1074) may be injection molded or welded to the tip (e.g., tip 450, 850, 1050A-D, and/or 1150A-B). For example, in some embodiments, the tip may be made of plastic (e.g., elastomer, high density thermoplastic elastomer, thermoplastic polyurethane, etc.) and injection molded over the distal end 212 of the shaft 210. In some embodiments, the tip may be made of metal and may be laser welded onto the distal end 212 of the shaft 210. Other materials (e.g., ceramics, alumina, etc.) and formation/attachment methods may also be used.

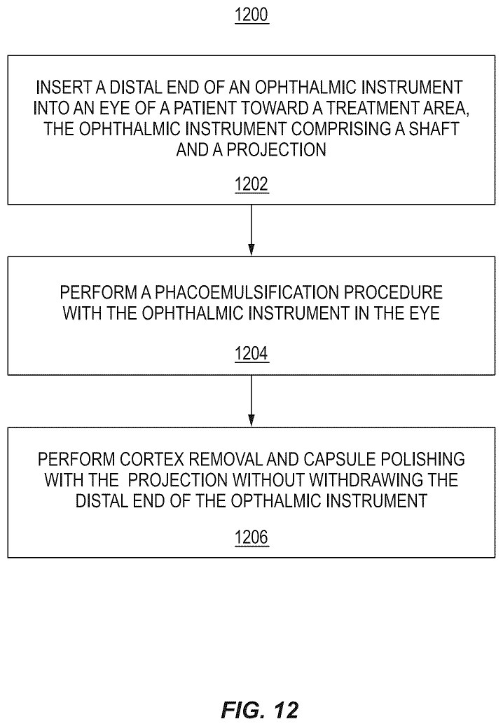

FIG. 12 illustrates a method 1200 for performing an ophthalmic surgical procedure (e.g., a phacoemulsification procedure), according to an embodiment. The elements provided in the flowchart are illustrative only. Various provided elements may be omitted, additional elements may be added, and/or various elements may be performed in a different order than provided below.

At block 1202, a distal end (e.g., distal end 802) of an ophthalmic surgical instrument (e.g., ophthalmic surgical instrument 801) may be inserted into an eye (e.g., eye 900) of a patient toward a treatment area. For example, the operator positions the distal end 802 of the ophthalmic surgical instrument 801 adjacent the treatment area such that the distal end 802 of the optical fiber 240 faces the treatment area (e.g., a lens of an eye) and such that the distal surface 856 of the projection 854 faces the treatment area.

At block 1204, a phacoemulsification procedure may be performed with the ophthalmic surgical instrument in the eye. For example, the phacoemulsification procedure can be performed with the ophthalmic surgical instrument 801 inserted in the eye 900. In some embodiments, during the phacoemulsification procedure, the operator delivers laser energy through an optical fiber (e.g., optical fiber 240) to the treatment area.

At block 1206, without withdrawing the distal end of the ophthalmic surgical instrument from the eye, cortex removal and capsule polishing may be performed with the projection.

In some embodiments, the method 1200 may further include reaching under a capsulotomy to facilitate removal of cortical material, including sub-incisional cortex. In some embodiments, the projection 854 may also be used as a hook to gently pull on the capsulotomy to further extend the projection's reach for removal of additional cortical material.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. An ophthalmic surgical instrument comprising:
a shaft having a distal end configured to be inserted into an eye of a patient toward a treatment area; and
a projection located at the distal end of the shaft, the projection having a distal surface that extends at an angle relative to a longitudinal axis of the shaft, the distal surface configured to be positioned facing the treatment area during use of the ophthalmic surgical instrument.

2. The ophthalmic surgical instrument of claim 1, wherein the projection further comprises grooves on an edge of the projection.

3. The ophthalmic surgical instrument of claim 1, wherein the projection further comprises one or more notches on the projection.

4. The ophthalmic surgical instrument of claim 1, wherein the projection further comprises at least one wing extending out of the projection.

5. The ophthalmic surgical instrument of claim 1, wherein the distal surface of the projection is a textured surface for polishing a lens capsule of the eye.

6. The ophthalmic surgical instrument of claim 1, further comprising a tip coupled to the shaft, wherein the projection is located on the tip.

7. The ophthalmic surgical instrument of claim 6, wherein the tip comprises:
an aspiration channel providing a pathway through the tip to the shaft, wherein material aspirated at the treatment area flows through the aspiration channel.

8. The ophthalmic surgical instrument of claim 7, wherein the aspiration channel is angled relative to the longitudinal axis of the shaft.

9. The ophthalmic surgical instrument of claim 1, wherein a surface area of the distal surface of the projection is at least two to eight times greater than a surface area of an end face of the shaft.

10. The ophthalmic surgical instrument of claim 1, further comprising an optical fiber for delivering laser energy to the treatment area, the optical fiber having a distal end configured to be positioned facing the treatment area during use of the ophthalmic surgical instrument.

11. The ophthalmic surgical instrument of claim 10, further comprising:
a tip coupled to the shaft, wherein the projection is located on the tip;
an aspiration channel providing a pathway through the tip to the shaft, wherein material aspirated at the treatment area flows through the aspiration channel;
wherein the optical fiber is disposed outside and adjacent the shaft, and
wherein the optical fiber enters the aspiration channel at the tip.

12. The ophthalmic surgical instrument of claim 1, further comprising an irrigation sleeve around the shaft, and wherein the projection is located on the irrigation sleeve.

13. The ophthalmic surgical instrument of claim 12, wherein the irrigation sleeve comprises a proximal hub configured to be coupled to a housing of the ophthalmic surgical instrument and a distal tube configured to fit around the shaft.

14. The ophthalmic surgical instrument of claim 1, wherein the shaft is tubular, and wherein the tubular shaft is configured for aspiration from the treatment area.
